(19) United States
(12) Patent Application Publication
(10) Pub. No.: US 2003/0109441 A1
(43) Pub. Date: Jun. 12, 2003

(54) Use of lactogenic and somatogenic hormones to enhance reproductive efficiency in mammals

(75) Inventors: Thomas E. Spencer, College Station, TX (US); Fuller W. Bazer, College Station, TX (US)

Correspondence Address: HOWREY SIMON ARNOLD & WHITE, 750 Bering Drive, Houston, TX, 77057-2198, US

(73) Assignee: Texas A&M University

(21) Appl. No.: 10/246,327

(22) Filed: Sep. 18, 2002

Related U.S. Application Data
(60) Provisional application No. 60/322,972, filed on Sep. 18, 2001.

(51) Int. Cl. A61K 38/24

(52) U.S. Cl. 514/12

(57) ABSTRACT

The present invention is generally directed to improving fertility in mammals. Methods of the present invention include increasing reproductive efficiency in mammals by administering at least one lactogenic or somatogenic hormone to a mammal in order to increase reproductive efficiency. Additional methods of the present invention include increasing endometrial adenogenesis in a mammal.

The U.S. government owns rights in the present invention pursuant to grant numbers HD-38274 and P30-ES-09106 from the National Institutes of Health and grant number US-3199-OCR from USDA BARD.

The present application claims the benefit of Provisional U.S. Application Serial No. 60/322,972, filed Sep. 18, 2001, which is herein incorporated by reference.

### FIELD OF THE INVENTION

The present invention relates generally to the field of mammalian fertility. More particularly, it concerns methods relating to increasing reproductive efficiency of a mammal by administering at least one lactogenic or somatogenic hormone to increase reproductive efficiency of the mammal.

### BACKGROUND OF THE INVENTION

The idea that uterine secretions play a role in nourishing the developing embryo is an ancient concept discussed by Aristotle in the third century BC. Subsequently, research has shown that all mammalian uteri contain endometrial glands that synthesize and either secrete or transport a complex array of proteins and related substances essential for survival and development of the conceptus, which is defined as the embryo or fetus along with the associated extraembryonic placental membranes. Evidence from studies of primate and subprimate species during the last century supports an important role for secretions of endometrial glands in many facets of reproduction including as primary regulators of conceptus survival, development, onset of pregnancy recognition signals, and implantation/placentation. Gray et al. (2001) provide a general review of this subject in their research article entitled “Developmental Biology of Uterine Glands”, which is hereby incorporated by reference in its entirety.

A wide variety of animal models have served as subjects for investigating uterine development and the role of endometrial glands and their secretions in this process. For example, in marsupials, carnivores, and roe deer, changes in endometrial gland secretory activity are believed to regulate delayed implantation. In rodents, several factors, including leukemia inhibitory factor and calcitonin, are produced exclusively by endometrial glands and are essential for the establishment of uterine receptivity and embryo implantation. Furthermore, uterine secretions are thought to be particularly important for conceptus survival and development in sheep, cattle, pigs, and horses, in which a prolonged period of pre-implantation conceptus development precedes superficial attachment and placentation.

In mammals, the uterus develops as a specialization of the paramesonephric or Mullerian ducts, which gives rise to the infundibulum, oviducts, uterus, cervix, and anterior vagina. The mature uterine wall is comprised of two functional compartments, the endometrium and the myometrium. The endometrium is the inner mucosal lining of the uterus and is derived from the inner layer of ductal mesenchyme. Histologically, the endometrium consists of two epithelial cell types, luminal epithelium (LE) and glandular epithelium (GE), which are two stratified stromal compartments including a densely organized stromal zone (stratum compactum) and a more loosely organized stromal zone (stratum spongiosum), blood vessels, and immune cells. The myometrium is the smooth muscle component of the uterine wall and includes an inner circular layer derived from the intermediate layer of ductal mesenchymal cells and an outer longitudinal layer derived from subperimetrial mesenchyme.

The development of all mammalian uteri include the following common morphogenetic events: 1) organization and stratification of endometrial stroma; 2) differentiation and growth of the myometrium; and 3) coordinated development of the endometrial glands.

Research by Gray et al. (2001), which is incorporated herein by reference in its entirety, indicates that early pregnancy failure and reductions in fetal survivability in livestock are due, in part, to inadequate development of the endometrial glands within the uterus. The process of uterine morphogenesis is governed by a variety of endocrine, cellular, and molecular mechanisms, but many of the details have not yet been elucidated. Similarly, the mechanisms regulating endometrial adenogenesis have also been unclear.

Previous medical research has focused on treating disease conditions involving the overproduction of prolactin by inhibiting the production of prolactin or by blocking its activity. An example of this type of treatment includes that described in U.S. Pat. No. 5,972,893, which describes methods for lowering abnormally high levels of prolactin in the blood of an animal.

Another aspect of prolactin activity is its ability to affect the immune system. U.S. Pat. No. 5,605,885 details methods for stimulating lymphocyte proliferation in humans with suppressed lymphocyte function using prolactin agonists. U.S. Pat. No. 4,837,202 describes methods for stimulating the immune system by increasing production of macrophages and for augmenting their oxidative metabolism by administering somatotropin or prolactin.

A further aspect of prolactin activity includes its effects on increasing tensile strength in connective tissue. U.S. Pat. No. 5,162,303 describes methods relating to improving the general condition of skin utilizing prolactin compositions and topical treatments.

### SUMMARY OF THE INVENTION

An embodiment of the present invention includes a method for increasing the reproductive efficiency of a mammal by administering an effective amount of at least one lactogenic hormone to increase the reproductive efficiency of the mammal.

Another embodiment of the present invention includes a method for increasing endometrial adenogenesis in a mammal by administering an effective amount of at least one lactogenic hormone to increase endometrial adenogenesis.

An additional embodiment of the present invention includes a method for increasing the reproductive efficiency of an adult mammal by administering an effective amount of at least one lactogenic or somatogenic hormone to a mammal to increase the reproductive efficiency of the mammal.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a panel of photomicrographs at two different magnifications of histological analyses of uteri from postnatal day (PND) 42 ewes, treated with vehicle as a control (CX), recombinant ovine prolactin (roPRL), recombinant ovine growth hormone (roGH), or recombinant ovine placental lactogen (roPL). Legend: Car, caruncle; LE, lumenal epithelium; M, myometrium: *, glands.

FIG. 2 shows histological analyses of uteri from one control (left panels) and two bromocryptine-treated (right panels) PND 56 ewes at two different magnifications. Legend: Car, caruncle; IE, intercaruncular endometrium; LE, lumenal epithelium; M, myometrium; *, glands.

FIG. 3 shows concentrations of prolactin (PRL; LSM±SEM) in serum from neonatal ewes implanted with a placebo pellet as a control (CX) or bromocryptine mesylate (BROMO) pellet from birth to PND 56.

FIG. 4 shows photomicrographs depicting effects of treatment with placebo pellets as a control (CX) or bromocryptine mesylate (BROMO) pellets from birth (PND 0) on uterine wall development at PND 56. Uterine tissue sections were prepared and stained with hematoxylin and eosin. Photomicrographs are shown at low (4X) magnification (top) with the area denoted by the white bar at a higher (20X) magnification (bottom). Note the reduction in coiled and branched endometrial glands in the stratum spongiosum of BROMO-treated ewes. Legend: Car, Caruncle; LE, lumenal epithelium; GE, glandular epithelium; Myo, myometrium.

FIG. 5 is a graph showing concentrations of prolactin (PRL; LSM±SEM) in serum from neonatal ewes treated with saline vehicle as a control (CX) or recombinant ovine prolactin (roPRL) from PNDs 1 to 56.

FIG. 6 is a graph depicting the uterine gland density in uteri from ewes treated with vehicle as a control (CX) or recombinant ovine PRL (roPRL) on PNDs 14 and 56. Ewes were assigned at birth to receive treatment with saline vehicle as a CX or roPRL from PNDs 1 to 55. On PND 14, ewes were hemi-ovariohysterectomized, and the remaining uterine horn and ovary removed on PND 56. Uterine tissue sections were prepared and stained with hematoxylin and eosin. Gland density was determined and is expressed as gland number per section (LSM±SEM).

FIG. 7 shows Western blots illustrating the effects of prolactin treatment of PND 28 ovine uteri on phosphorylation of STAT, ERK1/2, and SAPK/JNK proteins. Whole uterine explants from PND 28 ewes were treated with roPRL (500 ng/ml) for 0, 15, 30, 60, or 120 min., and 40 μg of each lysate was separated by SDS-PAGE and analyzed for phosphorylated STATs 1, 3, and 5 (shown in FIG. 7A); or ERK1/2 and JNK/SAPK (both shown in FIG. 7B) by Western blotting. Representative results from the analyses of uterine explants from five animals are shown. Positions of the prestained molecular weight markers are shown on the left.

### DETAILED DESCRIPTION OF THE INVENTION

The present invention provides novel methods relating generally to increasing fertility in mammals. These methods include increasing the reproductive efficiency of a mammal by administering an effective amount of at least one lactogenic hormone to increase the reproductive efficiency of the mammal. Another method includes increasing endometrial adenogenesis in a mammal by administering an effective amount of at least one lactogenic hormone to increase endometrial adenogenesis in the mammal. A further method includes increasing reproductive efficiency of an adult mammal by administering an effective amount of at least one lactogenic or somatogenic hormone to increase reproductive efficiency of the adult mammal.

In a particular embodiment, the methods according to the invention may include administering an effective amount of at least one lactogenic hormone and an effective amount of at least one somatogenic hormone. These hormones may be administered simultaneously or separately by any of the general procedures described herein.

A “lactogenic hormone” is a hormone that enhances the formation of milk. In a preferred embodiment, the lactogenic hormone is prolactin or placental lactogen.

A “somatogenic hormone” is a hormone that enhances growth. In a preferred embodiment, the somatogenic hormone is a growth hormone.

In a preferred embodiment, the lactogenic and somatogenic hormones used in the present invention may be recombinant hormones. In another preferred embodiment, the lactogenic and somatogenic hormones used in the present invention may be exogenous.

In the methods of the invention, the hormones may be administered by any conventional technique. The hormones may be administered directly into or onto a target tissue, or indirectly in such a manner that the hormone has a positive therapeutic impact on the tissue to which it is targeted. For example, the hormone(s) may be administered systemically, e.g., by intravenous injection whereby the hormone directly or indirectly reaches the target tissue, or by providing a gene therapy technique by which a genetic coding sequence is delivered to the mammal in order to express the desired hormone or to elicit an increase in production of the hormone by the pituitary and/or placenta. Other techniques for formulation and administration of pharmaceuticals may be found in the latest edition of Remington's Pharmaceutical Sciences (Mack Publishing Co, Easton Pa.). Although administration of hormones by injection is desirable, there are other means of delivery, for example: oral, parenteral, aerosol, intramuscular, subcutaneous, transcutaneous, intramedullary, intrathecal, intraventricular, intravenous, intraperitoneal, or intranasal administration. Various tablet formulations are also useful means of delivery, including time-release capsules.

The hormones may be administered to any site on the mammal that results in the desired effect. In a preferred embodiment, the hormone(s) may be administered directly to the uterus.

The methods according to the invention may be applied to mammals. In a preferred embodiment, the mammal is a human, a primate, or an ungulate. “Ungulates” are hoofed livestock, examples of which include sheep, cattle, goats and pigs. In a further preferred embodiment, the mammal is a sheep. In another further preferred embodiment, the mammal is a human.

The methods according to the invention may be applied at any life stage of the mammal. In a preferred embodiment, the hormone(s) are administered in utero to an embryo, to a neonatal mammal (an age defined herein as being from birth until about age 6 months), a pre-pubertal mammal, a pubertal mammal or to an adult mammal. In a further preferred embodiment, the hormone(s) are administered to a neonatal mammal, wherein the increased reproductive efficiency is particularly observed when the neonate reaches its reproductive years. Preferably the hormone(s) are administered from birth to about 90 days postnatally, and further preferably from birth to about 60 days postnatally.

In a preferred embodiment, the hormone(s) to be administered are given in an “effective amount.” As used herein, the term “effective amount” is used to mean any amount that will cause the desired result, such as increasing reproductive efficiency in the mammal, and/or increasing endometrial adenogenesis in the mammal, and/or enhancing endometrial adenogenesis or reproductive capacity in a uterus. In a preferred embodiment the hormone is administered in an amount of about 2 milligrams per kilogram body weight per day.

Methods of the present invention will also apply to situations wherein the uterus has been subject to at least one assisted reproductive technology. In a preferred embodiment, the assisted reproductive technology is in vitro fertilization followed by embryo transfer (IVF-ET). In a preferred embodiment, the uterus is an ungulate uterus and the hormone is a recombinant ovine prolactin. In another preferred embodiment, the method is applied to infertile female mammals, particularly humans, undergoing ovulation induction followed by in vitro fertilization or embryo transfer to enhance the rate of pregnancy. Administration of at least one lactogenic and/or somatogenic hormone according to the present invention may serve to increase endometrial adenogenesis of the subject's uterus and therefore provide a more favorable environment for implantation and further development of the embryo, ultimately resulting in increased fertility and overall healthier offspring.

In the present invention, the term “endometrial adenogenesis” refers to the process of endometrial gland development. In most mammals, this process involves extensive coiling and branching morphogenesis of the endometrium and ultimately is reflected in the glandularity of the adult uterus. Increasing endometrial adenogenesis refers to increasing the coiling and branching morphogenesis of the endometrium over that of an untreated subject, and will also result in higher numbers of glands present in the uterus, generally the formation of a more glandular uterus. In a preferred embodiment, the effects of increasing endometrial adenogenesis will be maintained throughout the reproductive life of the mammal.

In the present invention, the term “increasing the reproductive efficiency” refers to an increase in reproductivity over that of an untreated subject. Increased reproductive efficiency is exhibited by an increase in one or more of fetal-placental development rate, offspring birth weight, survivability of offspring, and number of offspring (either per pregnancy or number of pregnancies), healthier offspring and fewer miscarriages.

In specific illustrations, the present studies show that administering an effective amount of at least one lactogenic hormone to a neonatal ewe, result in increased endometrial adenogenesis and in the development of a more glandular uterus in the treated ewe. This increase in embryotrophic capacity and functional capacity will correspond to an increase in fetal-placental development rate, neonatal birth weight and neonatal survival. This treatment is also expected to enhance the embryotrophic and functional capacity of the adult uterus, resulting in fewer miscarriages and the birth of heavier and more vigorous offspring.

A preferred embodiment of this invention is to inject recombinant ovine prolactin (PRL) into neonatal female lamb (ewe). As used herein, the term ovine means of, relating to, or resembling sheep. The present invention will be particularly useful in the semi-intensive to intensive dairy sheep industries in the Mediterranean and European countries like Spain, Portugal, France, Italy, Greece, Turkey, Israel and Jordan where sheep milk is used extensively for the manufacture of cheese. Similar utility will also be found in the United States, Canadian, Australian, and New Zealand sheep industries. While the sheep industry is of particular interest, methods of the present invention will be useful in other livestock species, especially the ones with multiple births, such as goats and dairy, dual purpose and beef cattle.

Routine molecular techniques, such as those for nucleic acid manipulation are described generally, e.g., in Sambrook et al. Molecular Cloning: A Laboratory Manual, Cold Spring Harbor Press, Cold Spring, N.Y. (1989) or in Ausubel et al. Current Protocols in Molecular Biology, John Wiley & Sons, New York, N.Y. (1992).

The publications and other materials used herein to illuminate the background of the invention or provide additional details respecting the practice, are incorporated by reference, and for convenience are respectively grouped in the appended List of References.

Biological responses to PRL (prolactin) in mammalian model systems are mediated by the PRLR (prolactin receptor) and intracellular activation of several signal transduction systems, including signal transducers and activators of transcription (STAT) proteins 1, 3 and 5, interferon (IFN) regulatory factor one (IRF-1), and the mitogen activated protein kinase (MAPK) cascade, as described by Bole-Feysot et al. 1998; Freeman et al. 2000; and Yu-Lee 2001. In the human uterus, PRL is produced by the decidua and the PRLR is expressed both in the endometrial GE and stroma, as described by Jones et al. 1998 and Dalrymple and Jabbour 2000. Recent studies indicate that PRL stimulates extracellular regulated kinase (ERK) 1 as well as 2 MAPKs and STAT 1. PRL also increases interferon regulatory factor one (IRF-1) expression in primate and human endometrial glands as shown by Dalrymple and Jabbour 2000 and Jabbour et al. 1998. High levels of phosphorylated ERK 1 and 2 MAPKs are also detected in nascent and proliferating endometrial glands of the neonatal ovine uterus as described by Taylor et al. 2001.

Available data in the neonatal ewe and other model systems support the working hypothesis that the postnatal increase in circulating PRL activates PRLR signaling pathways in the nascent and proliferating endometrial GE to stimulate and maintain their coiling and branching morphogenesis in the neonatal ovine uterus. In order to test this hypothesis, the present studies were conducted in the neonatal ewe to determine: (1) effects of hypoprolactinemia on uterine adenogenesis; (2) effects of hyperprolactinemia on uterine adenogenesis; (3) effects of postnatal age on expression of STATs 1, 3 and 5 and IRF-1 proteins; and (4) if exogenous recombinant PRL stimulates STAT and MAPK signal transduction pathways in the developing uterus. Hypoprolactinemia is a condition of a lower than normal level of prolactin. Hyperprolactinemia is a condition in which an individual has an elevated level of prolactin, when compared to normal levels. Exogenous PRL refers to prolactin that is administered to the test subject. The exogenous prolactin (or) may be purified native prolactin from any suitable subject, or it may be recombinant prolactin produced by any suitable overexpression system. Similarly, any desired exogenous lactogenic or somatogenic hormone utilized in the present methods may be purified native hormone from any suitable subject, or it may be recombinant hormone produced by any suitable overexpression system.

Additionally, the prolactin may be provided to the subject by a transgenic technique by delivering the coding sequence of prolactin to the desired tissue, so that prolactin is subsequently expressed. These transgenic techniques may be applied to any desired lactogenic or somatogenic hormone to facilitate hormone expression in the desired tissue. In addition to direct injection and electroporation of DNA in vivo, there are numerous viral vector systems useful for these types of transgenic techniques; the application of such techniques for use in pigs is described in U.S. Pat. No. 6,271,436, which is herein incorporated by reference in its entirety. One system utilizes adenoviral vectors. Adenovirus growth and manipulation is known to those of skill in the art, and these viruses exhibit broad host range in vitro and in vivo, can be obtained in high titers, e.g., 10⁹-10¹¹ plaque-forming units per ml, and are highly infective. The life cycle of adenovirus does not require integration into the host cell genome. Thus, foreign genes, such as lactogenic or somatogenic hormones, delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors for genes such as those encoding lactogenic and/or somatogenic hormones as described in the present invention. In a specific example, Adeno-associated virus (AAV) would be an attractive vector system for use in the present invention since it has a high frequency of integration and it can infect nondividing cells, thus making it useful for delivery of genes into mammalian cells in tissue culture. Details concerning the generation and use of recombinant AAV vectors are described in U.S. Pat. No. 5,139,941 and U.S. Pat. No. 4,797,368, each incorporated herein by reference.

The following examples are included to demonstrate preferred embodiments of the invention. It will be appreciated by those of skill in the art that the techniques disclosed in the examples which follow represent techniques discovered by the inventor to function well in the practice of the invention, and thus can be considered to constitute preferred modes for its practice. However, those of skill in the art will, in light of the present disclosure, appreciate that many changes can be made in the specific embodiments which are disclosed and still obtain a like or similar results without departing from the spirit and scope of the present invention.

General Procedures and Reagents

The following general procedures and reagents were utilized in experiments in the following examples.

Care and Selection of Sheep

Crossbred Suffolk ewes were mated to Suffolk rams in the fall between the months of September and November. Pregnant ewes were maintained according to normal husbandry practices and fed hay and corn. Ewes used in Examples 3 through 6 were born in the spring between the months of February and May.

General Animal Care

All experiments and surgical procedures were conducted in accordance with the Guide for the Care and Use of Agriculture Animals and approved by the University Laboratory Animal Care Committee of Texas A&M University.

Antibodies

Antibodies used in the present study included: mouse anti-STAT 1 (#610185), mouse anti-STAT 3 (#610189) and mouse anti-STAT 5 (#610191) from BD Transduction Laboratories (Lexington, Ky.); rabbit anti-phospho-STAT 1 (#9171), rabbit anti-phospho-STAT 3 (Tyr 705; #9131), rabbit anti-phospho-STAT 5 (Tyr694) antibody (#9351), rabbit anti-phospho-p44/42 MAPK (Thr202/Tyr204) antibody (#9101), rabbit anti-p44/42 (ERK1/2) MAPK antibody (#9102), and rabbit anti-phospho-SAPK/JNK (Thr183/Tyr185) antibody (#9251) from Cell Signaling Technology (Beverly, Mass.); rabbit anti-human IRF-1 (sc-497) from Santa Cruz Biotechnology, Inc. (Santa Cruz, Calif.); peroxidase-labeled goat anti-mouse (#474-1806) and anti-rabbit IgG (#474-1506) from Kirkegaard & Perry Laboratories (Gaithersburg, Md.); and normal rabbit IgG (#15006) and normal mouse IgG (#15381) from Sigma-Aldrich (St. Louis, Mo.).

Preparation of Recombinant Ovine Prolactin

Recombinant ovine PRL (roPRL) (GenBank Accession No. M27057) was prepared in Escherichia coli cells as described by Leibovich et al. (2001) which is hereby incorporated by reference in its entirety. The expressed protein, found in inclusion bodies, was refolded and purified to homogeneity on a Q-Sepharose column, yielding an electrophoretically pure fraction composed of over 98% monomeric protein of the expected molecular mass of approximately 23 kiloDaltons (kDa). The biological activity of the roPRL after proper renaturation was evidenced in vitro by its ability to stimulate proliferation of rat lymphoma Nb2 cells possessing prolactin receptor (PRLR), to stimulate luciferase activity in human embryonic kidney 293 cells (HEK 293) transiently transfected with ovine PRLR, and to induce progesterone secretion in primary cultures of luteal cells obtained from midpregnant ewes as described in Leibovich et al. 2001

Histology

After 24 hours (h) of fixation, uterine tissues were changed to 70% ethanol for 24 h and then dehydrated and embedded in Paraplast Plus (Oxford Labware, St. Louis, Mo.). Uteri were sectioned (4-6 micrometers (μm)) and stained with hematoxylin and eosin as described previously by Gray et al. 2000, which is incorporated herein by reference in its entirety. Relative endometrial gland density was determined by counting the total number of uterine glands in a complete cross-section of the ovine uterine horn using methods similar to those described in Spencer et al. 1999, which is incorporated herein by reference in its entirety. Gland density estimates were generated for at least 10 nonsequential sections from each uterine horn. The observation of a gland cross-section with a visible open lumen was counted as a gland. Intra- and inter-section repeatability estimates for determination of gland number by a single observer was 0.85 and 0.8, respectively. Data are presented as gland numbers per uterine horn cross-section.

Immunohistochemistry

Expression of immunoreactive STAT 1, STAT 3, STAT 5 and IRF-1 proteins was detected in uterine tissue cross-sections (5-7 μm) using the appropriate mouse STAT antibodies and rabbit IRF-1 antibody and a Super ABC Mouse/Rat IgG Kit (Biomeda, Foster City, Calif.) according to methods described previously by Spencer and Bartol et al. 1999, which is hereby incorporated by reference in its entirety. The mouse antibodies detect both unphosphorylated and phosphorylated forms of the STAT proteins. The final working antibody dilutions were 1:1000 for STAT antibodies and 1-20 μg/ml for IRF-1. Antigen retrieval utilizing boiling citrate buffer was performed as described previously by Taylor et al. 2000 and Taylor et al. 2001, each of which is incorporated by reference in its entirety herein. The chromagen used for peroxidase localization was 3,3′-diaminobenzidine tetrahydrochloride from Sigma. Negative controls were performed in which the primary antibody was substituted with the same concentration of purified normal mouse IgG or normal rabbit IgG from Sigma Chemical Co. (St. Louis, Mo.). Multiple tissue sections from each ewe were processed as sets within an experiment.

Radioimmunoassay (RIA)

Blood samples were allowed to clot for 1 h at room temperature. Serum was then collected by centrifugation (3000×g for 30 min at 4° C.), removed and stored at −20C. for hormone analyses. Concentrations of PRL in serum were determined using reagents for the ovine PRL RIA provided by Dr. A. F. Parlow and the National Institute of Diabetes and Digestive and Kidney Diseases (NIDDK) National Hormone and Pituitary Program as described previously in Taylor et al. 2000. Purified ovine PRL (NIDDK-oPRL-I-3) was iodinated using the chloramine T reaction, and the assay conducted using methods and reagents provided by the NIDDK Pituitary Hormones and Antisera Center. Assay sensitivity was 0.1 ng/ml, and the intra- and inter-assay coefficients of variation were 5% and 12%, respectively. Concentrations of estradiol-17β in the serum were determined using reagents for the estrogen RIA as described previously in Taylor et al. 2000. Assay sensitivity was 1 picogram (pg) per tube, and the intra-assay and interassay coefficients of variation were 8% and 14%, respectively. Assay results were calculated using the AssayZap Version 3.1 program (Biosoft, Ferguson, Calif.).

Photomicroscopy

Photomicrographs were taken using a Nikon Eclipse 1000 photomicroscope (Nikon Instruments Inc., Lewisville, Tex.) fitted with a Nikon DXM1200 digital camera. Digital images were captured and assembled using Adobe Photoshop 5.5 (Adobe Systems, Seattle, Wash.).

Western Blot Analyses

The protein concentration of the supernatant was determined by Bradford assay (Bio-Rad Laboratories, Burlingame, Calif.) using BSA as the standard. Forty (40) micrograms of proteins from each uterine explant were separated by SDS-PAGE and transferred to nitrocellulose as described previously in Spencer et al. 1999. Blots were blocked for 1 h at room temperature with either 5% w/v bovine serum albumin, TrisH: buffered saline, 0.1% Tween-20 (5% BSA-TBST) for phospho-specific antibodies or 5% non-fat milk-TBST for all other antibodies. Primary antibodies were diluted according to manufacturers recommendations in either 5% BSA-TBST for phospho-specific antibodies or 2% milk-TBST for all other antibodies. Blots were incubated with primary antibody overnight at 4° C., rinsed for 30 min at room temperature with TBST, incubated with the appropriate peroxidase-conjugated secondary antibody for 1 h at room temperature, and then rinsed again for 30 min at room temperature with TBST. Immunoreactive proteins were detected using enhanced chemiluminescence (SuperSignal West Pico Luminol System, Pierce, Rockford, Ill.) according to the manufacturer's recommendations using X-OMAT AR film (Kodak, Rochester, N.Y.). For loading control, Western blots probed with phospho-specific antibodies were reprobed with antibodies that detected both phosphorylated and unphosphorylated protein.

Statistical Analyses

All quantitative data were subjected to least-squares analysis of variance (LSANOVA) using General Linear Models (GLM) procedures of the Statistical Analysis System, as described in the SAS User's Guide 1990. In all analyses, error terms used in tests of significance were identified according to the expectation of the mean squares for error. Data are presented as least-square means (LSM) with overall standard errors (SE).

### EXAMPLE 1

### Effects of Exogenous Recombinant Ovine Prolactin (roPRL), Placental Lactogen (roPL) and Growth Hormone (roGH) Administration on Endometrial Gland Morphogenesis in the Neonatal Ewe

Experiments administering exogenous recombinant ovine prolactin (roPRL), placental lactogen (roPL) and/or recombinant growth hormone (roGH) were conducted to determine whether these procedures would be viable noninvasive methods to increase endometrial adenogenesis in the neonatal ewe. Sixteen Suffolk ewe lambs (n=4 ewes per treatment) received saline vehicle (CX) as a control, roPRL, roPL or roGH from postnatal day (PND) 14 to PND 55. Note that birth is PND 0. All recombinant hormones were prepared in saline and given at a dose of 2 mg per day.

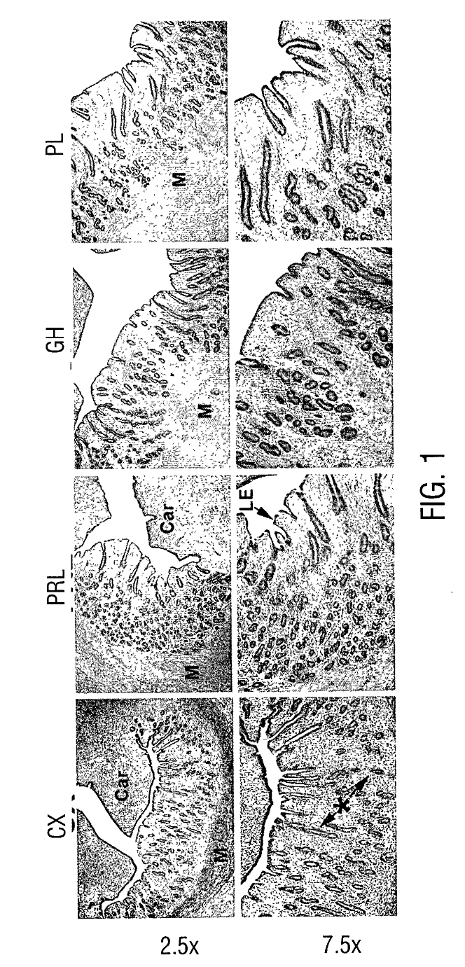

All ewes were hysterectomized on PND 56. Cross-sections from the middle of each uterine horn were fixed, embedded in paraffin, sectioned (4-5 μm) and stained with hematoxylin and eosin. The number of endometrial glands was determined by counting the total number of gland cross-sections with a visible lumen in sections of the uterine horn. A minimum of twenty uterine horn sections was quantitated per ewe and horn. Data was analyzed by least squares analysis of variance and is presented as least squares means (LSM) with overall standard errors (SE). Treatment effects were elucidated using pre-planned orthogonal contrasts (CX vs. roGH, CX vs. roPL, CX vs. roPRL). Data representative of the results achieved are presented in FIG. 1.

The results in FIG. 1 show that treatment with PRL increased (P<0.05) the number of uterine endometrial glands (CX vs. roPRL: 519±52 vs. 875±52 glands per section). However, treatment with either roGH (623±52 glands per section) or roPL (625±52 glands per section) appears to have an insignificant effect (P>0.10) on the number of uterine endometrial glands. When compared to CX ewes, uteri of roPRL-treated ewes consistently contained more coiled and branched uterine glands in the lower stroma near the myometrium. The number of endometrial folds and gland invaginations present at the lumenal surface were not affected by treatment. The myometrium appeared thicker in the uteri of GH-treated ewes but not in uteri from roPRL- or roPL-treated ewes. Similar experiments on adult GH-treated ewes showed an increase in the number of uterine endometrial glands, as described in Spencer and Stagg et al. 1999, which is incorporated herein by reference in its entirety.

### EXAMPLE 2

Inhibition of PRL Secretion by Bromocryptine Administration Suppresses Endometrial Adenogenesis in the Neonatal Ewe

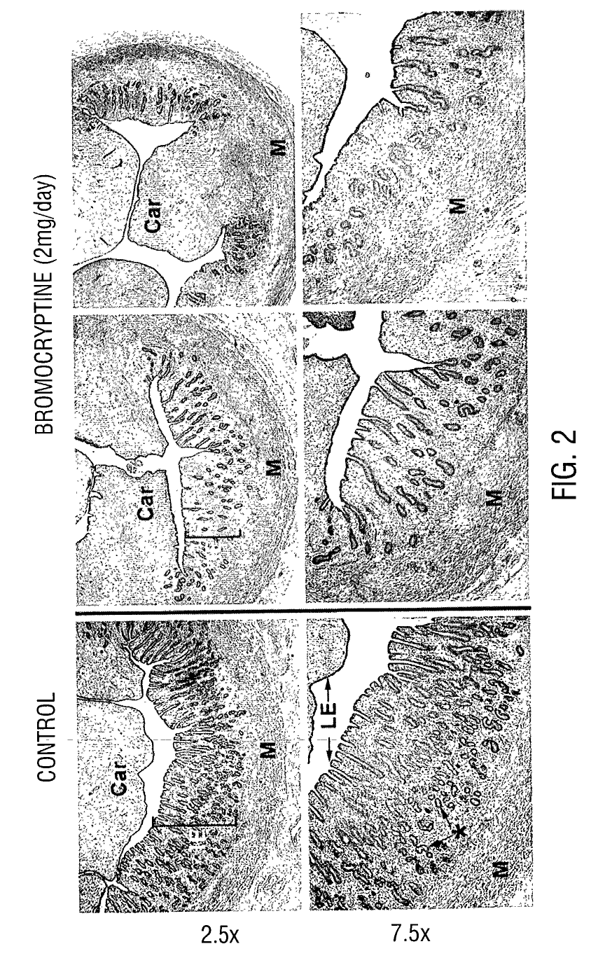

Neonatal ewes (n=5 per treatment) were assigned randomly at birth (postnatal day or PND 0) to receive twice daily injections of either phosphate-buffered saline acidified with 0.1 M tartaric acid as a control (CX), or 2 mg bromocryptine from PND 7 to PND 41. Bromocryptine is a dopamine D2 receptor agonist that inhibits the release of PRL from the pituitary. Blood samples were taken via jugular venipuncture every 7 days. All ewes were hysterectomized on PND 42. The mid-portion of each uterine horn was fixed in 4% paraformaldehyde, embedded in paraffin, sectioned (4-5 μm) and then stained with hematoxylin and eosin. Serum PRL levels were determined by RIA using reagents provided by the NIDDK. Data representative of the results achieved are shown in FIG. 2.

As illustrated in FIG. 2, the folded intercaruncular endometrium of control uteri contained large numbers of coiled and branched endometrial glands which were terminally differentiated near the inner circular layer of myometrium. In contrast, the endometrium from bromocryptine-treated ewes contained far fewer glands. Although some tubular glands were present, the endometrium of bromocryptine-treated ewe lambs lacked the characteristically branched and terminally differentiated glands proximal to the myometrium that were present in control uteri. In addition, it should be noted that intercaruncular endometrial folds and the number of gland invaginations present at the lumenal surface were noticeably reduced by bromocryptine treatment. It was also noted that no distinct treatment effects were detected on caruncular areas of the endometrium or myometrium. Serum PRL concentrations were 307±25 ng/ml in PND 42 control ewes as compared to 50±10 ng/ml in bromocryptine-treated ewes.

Collectively, the results from Experiments 1 and 2 indicate that the hormone prolactin (PRL) is an important regulator of endometrial gland morphogenesis in the neonatal ovine uterus. We have discovered that treatment of neonatal ewes with exogenous recombinant ovine prolactin (roPRL) enhanced endometrial gland number and development (see FIG. 1) and inhibition of PRL secretion by bromocryptine resulted in a corresponding decrease in gland number and development (see FIG. 2). Thus, it appears that endometrial adenogenesis in the developing ovine uterus requires high circulating levels of PRL acting via the prolactin receptors (PRL-R), which are expressed by nascent and proliferating uterine glandular epithelium, to promote and maintain cell proliferation, differentiation and tubular branching morphogenesis. These results indicate that the administration of exogenous roPRL is effective in increasing endometrial glandularity in the neonatal ewe.

### EXAMPLE 3

### Hypoprolactinemia Retards Endometrial Adenogenesis

Uterine endometrial adenogenesis in the neonatal ewe is coincident with a postnatal rise in circulating levels of PRL and exclusive expression of PRLR in nascent and proliferating endometrial glands as shown in Taylor et al. 2000. The present study was conducted to test the hypothesis that lowering circulating levels of PRL with bromocryptine mesylate, a dopamine D2 receptor agonist and inhibitor of PRL secretion in the ewe, would retard or prevent endometrial adenogenesis. Ewe lambs received a biodegradable pellet that released placebo vehicle as a control (CX) or bromocryptine mesylate (BROMO) every 20 days beginning at birth.

Biodegradable placebo and bromocryptine mesylate (100 mg) 21-day release pellets were obtained from Innovative Research of America (Sarasota, Fla.). Ten ewe lambs (n=5 per treatment) were assigned randomly at birth (postnatal day or PND 0) to be implanted with a placebo pellet as a control (CX) or 100 mg bromocryptine mesylate pellet (BROMO) that releases 100 mg over a 21-day period, approximately 4.8 mg per day, to determine effects on uterine gland development. Biodegradable pellets were placed subcutaneously in the periscapular region every 20 days from birth. Blood samples were collected every 7 days beginning at birth by jugular venipuncture. On PND 56, all ewes were hemi-ovariohysterectomized. For removal of the right uterine horn and ovary, a hemostat was clamped perpendicular across the uterine horn at bifurcation of the uterine horns. A scalpel blade was used to remove the right uterine horn, oviduct and ovary. Electrocautery was used to seal the opening of the remaining portion of the uterine horn. The uterine horn piece was then trimmed free of the broad ligament, oviduct and cervix. Sections (˜1 cm) from the mid-portion of the uterine horn were fixed in fresh 4% paraformaldehyde in PBS (pH 7.2) for 24 h at room temperature and processed for histology as described below.

Circulating levels of PRL were affected by treatment (P<0.0001) and day (P<0.10), but not their interaction (FIG. 1). In CX ewes, serum levels of PRL were high on PND 1, increased to a maximum on PND 14, and decreased thereafter (cubic, P<0.10). Overall, circulating levels of PRL were 4.5-fold lower (P<0.0001, treatment) in BROMO than CX ewes. In BROMO ewes, serum PRL levels were much lower on PND 1 and increased two-fold (P<0.10, linear) to PND 56, but always remained much lower than PRL levels in CX treated ewes. Treatment with BROMO did not affect (P>0.10) serum levels of estradiol-17β compared to the level in CX treated ewes.

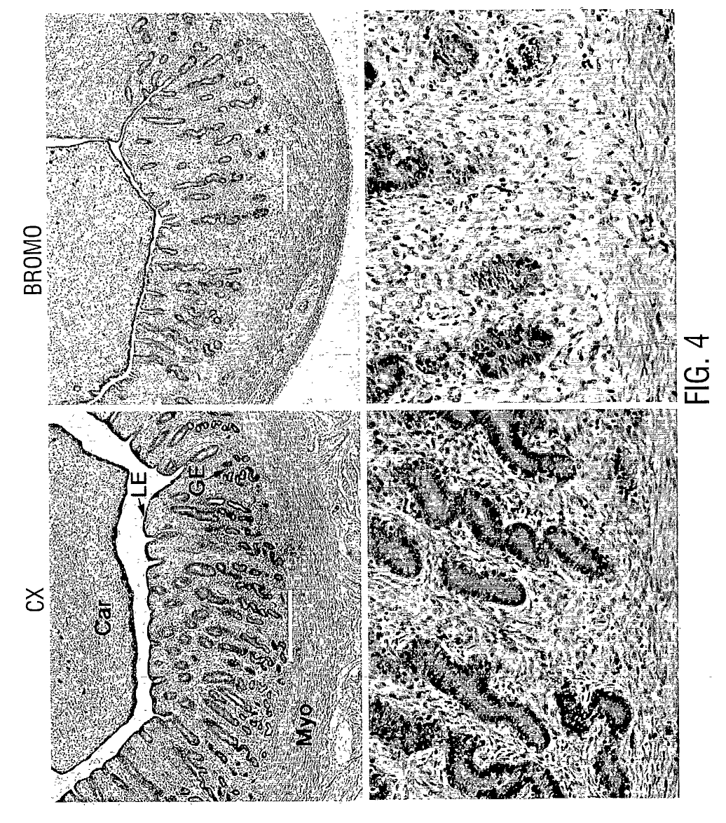

Histological analyses of the uterine wall indicated that the endometrium of CX ewes contained large numbers of coiled and branched glands in the intercaruncular endometrium as shown in FIG. 4. In contrast, the endometrium of BROMO ewes lacked the large numbers of characteristically coiled and branched glands in lower stroma of CX uteri. Histomorphometrical analyses indicated that treatment of neonatal ewes with BROMO decreased (P<0.01) endometrial gland density by 35% (CX vs BROMO: 695±30 vs 455±30 glands per section).

Hypoprolactinemia Retards Endometrial Adenogenesis

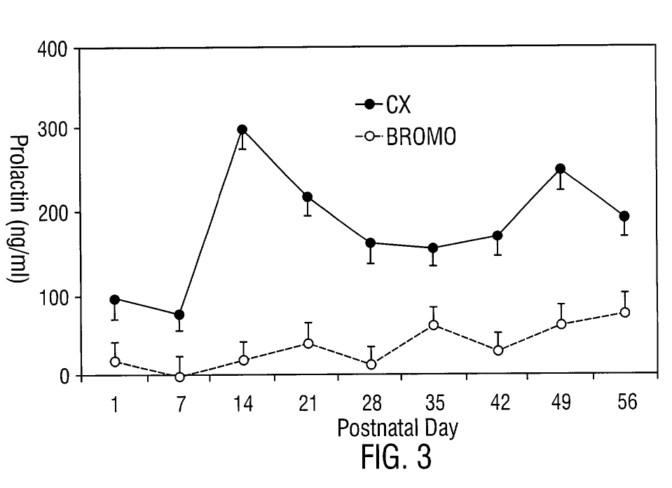

Uterine endometrial adenogenesis in the neonatal ewe is coincident with a postnatal rise in circulating levels of PRL and exclusive expression of PRLR in nascent and proliferating endometrial glands. The present study was conducted to test the hypothesis that lowering circulating levels of PRL with bromocryptine mesylate, a dopamine D2 receptor agonist and inhibitor of PRL secretion in the ewe, would retard or prevent endometrial adenogenesis. Ewe lambs received a biodegradable pellet that released placebo vehicle as a control (CX) or bromocryptine mesylate (BROMO) every 20 days beginning at birth. Circulating levels of PRL were affected by treatment (P<0.0001) and day (P<0.10), but not their interaction (FIG. 3). In CX ewes, serum levels of PRL were high on PND 1, increased to a maximum on PND 14, and decreased thereafter (cubic, P<0.10). Overall, circulating levels of PRL were 4.5-fold lower (P<0.0001, treatment) in BROMO than CX ewes. In BROMO ewes, serum PRL levels were much lower on PND 1 and increased two-fold (P<0.10, linear) to PND 56, but always remained much lower than CX ewes. Treatment with BROMO did not affect (P>0.10) serum levels of estradiol-17β compared to CX ewes.

Histological analyses of the uterine wall indicated that the endometrium of CX ewes contained large numbers of coiled and branched glands in the intercaruncular endometrium, as shown in FIG. 4. In contrast, the endometrium of BROMO ewes lacked the large numbers of characteristically coiled and branched glands in lower stroma of CX uteri. Histomorphometrical analyses indicated that treatment of neonatal ewes with BROMO decreased (P<0.01) endometrial gland density by 35% (CX vs BROMO: 695±30 vs 455±30 glands per section).

### EXAMPLE 4

### Hyperprolactinemia Increases Endometrial Gland Development

Hyperprolactinemia elicits uterine glandular hyperplasia in the adult mouse, rabbit and pig, as shown by Chilton et al. 1988, Young et al. 1989, and Kelly et al. 1997; each of which is incorporated by reference herein in its entirety. In order to test this hypothesis in the neonatal ewe model, ewes in the present study were treated daily from birth to PND 55 with either saline vehicle as CX or roPRL (2 mg/kg body weight; a representative effective amount for ewes). Ewes were hemi-ovariohysterectomized on PND 14, and the remaining uterine horn and ovary was removed on PND 56.

Crossbred Suffolk ewes (n=5 per treatment) were randomly assigned at birth (PND 0) to receive twice daily injections (0700 h and 1800 h) of sterile saline vehicle as a control (CX) or roPRL (1 mg per kg body weight) from PND 1 to 55 to determine effects on uterine gland development. Body weight of the ewes was determined every 4 days and used to adjust treatments. Blood samples were collected every 8 days beginning on PND 1 by jugular venipuncture. On PND 14, all ewes were subjected to mid-ventral laparotomy. The right ovarian pedicle was ligated, and the ovary and oviduct removed. One-half of the ipsilateral anterior uterine horn was then removed and fixed in fresh 4% paraformaldehyde in PBS (pH 7.2) and processed for histology as described below. On PND 56, all ewes were weighed and necropsied. The left ovary was trimmed free of the mesovarium and weighed. The uterus was obtained and trimmed free of the broad ligament, oviduct and cervix. The entire left uterine horn was dissected free of the partial left uterine horn and weighed. Sections (˜1 cm) from the mid-portion of the uterine horn were fixed in fresh 4% paraformaldehyde in PBS (pH 7.2) and processed for histology as described below.

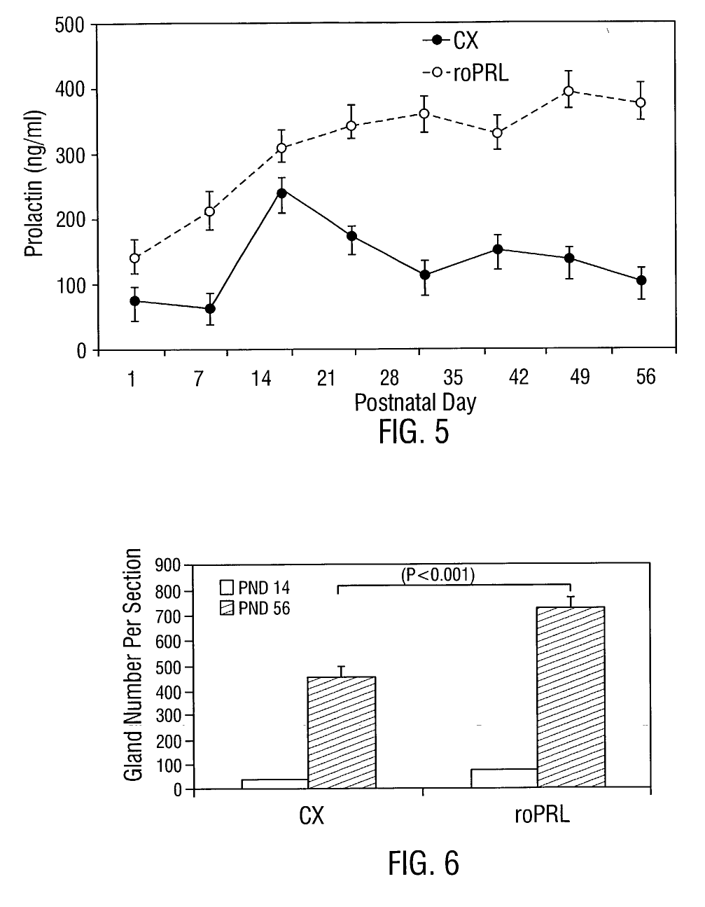

Serum levels of PRL were affected (P<0.01) by day, treatment and their interaction, as shown in FIG. 5. In CX ewes, serum levels of PRL were high on PND 1, reached a maximum on PND 17, and decreased thereafter (cubic effect of day, P<0.05). Overall, treatment of neonatal ewes with roPRL increased circulating levels of PRL (P<0.01, treatment). In roPRL ewes, serum levels of PRL were higher than CX ewes on PND 1 (P<0.01, day x treatment) and increased between PNDs 1 and 56 (quadratic effect of day, P<0.10). Treatment with roPRL did not affect (P>0.10) serum levels of estradiol-17β as compared to CX ewes. On PND 56, ovarian weight was not affected (P>0.10) by treatment (CX vs roPRL: 1.2±0.1 vs 0.8±0.1 g). Weight of the left uterine horn was also not affected (P=0.74) by treatment (CX vs roPRL: 2.3±0.2 vs 2.1±0.2 g).

Treatment of neonatal ewes with roPRL affected uterine gland morphogenesis. On PND 14, the endometrium of CX ewes contained nascent glands that were mostly tubular. On PND 56, the endometrium of CX ewes contained large numbers of coiled and branched endometrial glands, particularly in the stratum spongiosum endometrium. Treatment of neonatal ewes with PRL from birth did not affect endometrial gland development on PND 14, but increased the number of endometrial glands in the lower stratum spongiosum on PND 56.

Histomorphometrical analyses indicated that the number of endometrial glands was affected (P<0.01) by day, treatment and their interaction, as shown and summarized in the bar graph of FIG. 6. In CX ewes, endometrial gland density increased 7.4-fold (P<0.001) from PND 14 to PND 56 (61±7 vs 450±32). Administration of roPRL did affect (P=0.09) endometrial gland density on PND 14 (CX vs roPRL: 36±7 vs 61±7 total glands per uterine section), but increased (P<0.01) endometrial gland density on PND 56 by 63% (CX vs roPRL: 450±32 vs 732±32 glands).

### EXAMPLE 5

### Immunoreactive STATs 1, 3 and 5 are Present in the Developing Endometrial Glands

In other model systems, the biological effects of PRL mediated by the PRLR activate an intracellular signaling pathway involving ERK 1 and 2 MAPKs, STATs 1, 3 and/or 5, and IRF-1. Abundant levels of phosphorylated ERK 1 and 2 MAPKs are present in the nascent and developing glands of the neonatal ovine uterus. Therefore, the present study determined effects of postnatal age on expression of STATs 1, 3 and 5 as well as IRF-1 in the developing ovine uterus.

Ewes were assigned randomly at birth (PND 0) to be necropsied (n=5 ewes per day) on PNDs 1, 7, 14, 28, 42 or 56 to examine temporal and spatial alterations in expression of STATs 1, 3 and 5. At necropsy, the entire reproductive tract was excised, and the uterus was trimmed free of the broad ligament, oviduct and cervix. Cross-sections from the mid-portion of each uterine horn were fixed in 4% paraformaldehyde in PBS (pH 7.2) and processed for histology as described below.

STAT 1 protein was detected in all cell types on PND 1, but was more abundant in the LE. On PND14 and thereafter, STAT 1 protein was detected the developing GE. In the stroma, STAT 1 protein expression declined with age, but was detected in the nascent and proliferating glands. STAT 3 protein was detected in the LE and stroma. Expression of STAT 3 protein was particularly abundant in the LE and nascent and developing GE. STAT 5 protein was detected in all uterine cell types on PND 1, but was most abundant in the stroma and LE. STAT 5 protein was detected in the nascent and developing GE throughout development. Negligible levels of background were detected in negative controls wherein the primary antibodies were replaced with an equal amount of non-specific rabbit IgG.

Although IRF-1 protein was detected in immune cells, immunoreactive IRF-1 protein was not detected in the endometrial glands or in any other uterine cell types regardless of neonatal age. In situ hybridization analyses of the neonatal ovine uterus also confirmed these results using a homologous full-length ovine IRF-1 cRNA probe and methods described previously by Choi et al. 2001, which is incorporated herein by reference in its entirety. In all uterine cell types, IRF-1 mRNA was not detected, but present in immune cells. These studies show that STATs 1, 3, and 5 are present in the developing ovine uterus and, in particular, are expressed in the nascent and developing endometrial glands.

### EXAMPLE 6

### Prolactin Stimulates Phosphorylation of STATs 1 and 5, ERK 1 and 2, and JNK/SAPK

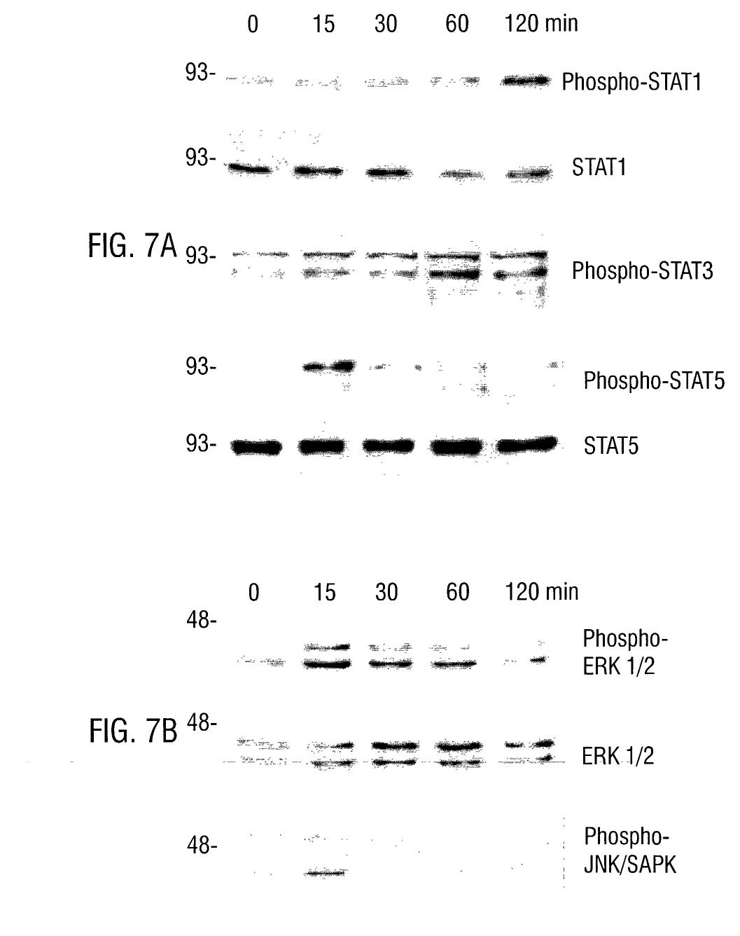

In order to investigate PRLR signaling in the neonatal ovine uterus, uteri from PND 28 ewes were explanted in serum-free medium and stimulated with 500 ng/ml of purified native oPRL. The explants were harvested at specific times, and the effects of PRL on STAT, ERK 1 and 2 MAPK, and JNK/SAPK signaling pathways were determined by Western blot analysis of proteins isolated from whole uterine explants as shown in FIG. 7. Treatment of uterine explants with PRL elicited a transient increase in tyrosine phosphorylated STAT 5 at 15 and 30 min post-treatment, but had no effect on STAT 3, as illustrated in FIG. 7A.

Five ewes were hysterectomized on PND 28 for uterine explant cultures. The uterus was trimmed free of the broad ligament, oviduct and cervix and placed in Dulbecco's modified eagle's medium with F-12 salts (DMEM-F12; Sigma-Aldrich, St. Louis, Mo.) supplemented with antibiotic-antimycotic (Gibco-BRL). The uterus from each ewe was weighed (˜3 g). In a laminar flow hood, uterine horns were separated and opened along the mesometrial side with a pair of fine surgical scissors. Uteri were then cut into small pieces (4-5 mm³), and explants placed in a 150-mm culture dish (B-D Labware, Franklin Lakes, N.J.) containing 50 ml of culture medium (serum-free DMEM/F12 with antibiotic-antimycotic). Uterine explants were cultured in a rocking Bellco incubator (Bellco Glass Inc., Vineland, N.J.) at 37° C. in an atmosphere of 5% CO₂/95% O₂ for 3 to 4 h. The culture medium was then replaced with fresh medium. Uterine tissue (300 mg) was then placed in a 60×15-mm culture dish (B-D Labware) containing 3 ml of culture media with 500 ng/ml of purified ovine pituitary PRL (NIDDK-oPRL-21) from Dr. A. F. Parlow at the NIDDK-NIH. Explants were placed in a tissue culture incubator and cultured at 37° C. in an atmosphere of 5% CO₂/95% O₂ for 0, 15, 30, 60 or 120 min.

At the designated time, uterine tissue was removed from the culture dishes, blotted on sterile gauze, and placed in 2 ml of freshly prepared, ice-cold lysis buffer (60 mM Tris (pH 6.8), 1 mM sodium orthovanadate, 10% glycerol, 2% SDS, aprotinin (44 μg/ml) and PMSF (100 μg/ml). Tissue was homogenized using a PRO250 homogenizer (Pro Scientific Inc., Monroe, Conn.) and then ground using a 2 ml Dounce tissue grinder (Kontec Glassware Company, Vineland, N.J.) with 30 strokes of the B pestle. Tissue homogenate was then clarified by centrifugation for 5 min at 20,000×g at 4° C. The supernatant was aliquoted and frozen at ˜80° C. for Western blot analysis.

The phospho-specific antibody used for this study detects both STAT 5a and 5TAT 5b. Levels of tyrosine phosphorylated STAT 1 were also increased by PRL between 60 and 120 min post-treatment. Within 15 min, PRL stimulated an increase in threonine and tyrosine phosphorylation of ERK 1 and 2 MAPKs as illustrated in FIG. 7B. Similarly, PRL also stimulated an increase in threonine and tyrosine phosphorylation of JNK/SAPK by 15 min post-treatment.

The results from example 6 show that PRL was observed to increase phosphorylation of STATs 5 and 1, but not STAT 3 in uterine explants from a PND 28 ewe. Interestingly, the effect of PRL on phospho-STAT 1 levels was more protracted and not observed until 60 min. Activation of the JAK2/STAT 5 cascade by PRL probably represents the hallmark of PRL signaling. Functional development of the mammary gland epithelium during pregnancy depends on PRL signaling, and STAT 5a is essential for mammary gland alveolar proliferation and function. Therefore, PRL signaling via the PRLR and STAT 5 are important for endometrial adenogenesis in the uterus during the neonatal period. However, the precise roles of STAT 5 are not known in neonatal ovine uterine gland development or in adult uterine gland hyperplasia and hypertrophy that normally occurs in response to PL in pregnant ewes.

Results from the above-described studies demonstrate that PRL regulates the critical process of endometrial gland morphogenesis in the developing uterus of the neonatal ewe. Serum PRL levels in CX ewes in both examples 2 and 3 were relatively high on PND 1, reached a maximum around PND 14, and then declined. Serum levels of PRL on PND 56 are much greater than in adult ewes during most of the estrous cycle and pregnancy. The temporal changes in circulating levels of PRL in the neonatal ewe parallel the ontogeny of endometrial glands in the developing intercaruncular endometrium of the uterine wall. Between PND 1 and 7, the endometrial GE buds from the LE and begins expressing the PRLR gene. After PND 7, the PRLR is expressed exclusively in endometrial GE and is highest in active glands that are proliferating and undergoing morphogenic development in the lower stratum spongiosum after PND 14.

Results of the present study and the fact that PRLR gene expression has been documented in endometrial GE of sheep, primates and humans, during periods of hyperplasia and hypertrophy of GE, indicate that PRL and PRLR interaction is a key mechanistic component that regulates endometrial morphogenesis and uterine gland development in both prepubertal and adult mammals. In Spencer et al. (1999), studies showed that adult ewes treated with placental lactogen or growth hormone showed increased endometrial adenogenesis and glandularity of the uterus in the treated adult ewes. In sheep and rodents, secretory products of the endometrial glands are required for conceptus survival and implantation. Further, Burton et al. 2002, have provided evidence that endometrial glands are an important source of nutrients for the human fetus during the first trimester when metabolism is essentially anaerobic. The success of developmental events regulating endometrial gland morphogenesis ultimately determines the functional capacity and embryotrophic potential of the adult uterus of mammals such as livestock and humans. Therefore, high and unexplained rates of peri-implantation embryonic losses in mammals such as livestock and humans may reflect, in part, unrecognized defects in endometrial adenogenesis or function induced during critical organizational periods in the neonate or adult. In women and menstruating primates, the long pre- and peri-pubertal period during which endometrial adenogenesis occurs, and the cyclical nature of adult endometrial regeneration, provide significant and repeated opportunities for endometrial dysgenesis and development of pathological lesions that could contribute to infertility. Results from the present study in sheep and others in humans show that PRL is an important regulator of endometrial gland morphogenesis and function in the uterus. Given the central importance of uterine glands and their secretions to support early embryonic survival and development, it is likely that perturbation of PRL secretion or the PRLR in the endometrium could lead to early pregnancy loss or infertility. Indeed, increased knowledge of endometrial gland development and function will lead to therapies for enhancing the low success rate of assisted reproductive technologies in humans.

In view of the above examples, one of ordinary skill in the art can appreciate that the present invention encompasses a method to increase reproductive efficiency in mammals, including specifically, ungulate livestock. Such a method includes administering at least one exogenous lactogenic and/or somatogenic hormone immediately after birth to a neonate in an amount sufficient or effective to increase reproductive efficiency, or endometrial adenogenesis, or both processes, resulting in the adult uterus having enhanced embryotrophic potential and functional capacity. Such a method includes the step of administering a sufficient or effective amount of exogenous lactogenic and/or somatogenic hormone to enhance the embryotrophic potential and functional capacity of the resulting fully developed adult uterus. An embodiment of the present invention includes the compositional mixture administered to a mammal in an amount sufficient to enhance the embryotrophic potential and functional capacity of the fully developed adult uterus.

Additional embodiments of the invention include administering a combination of PRL and GH to a mammal to increase endometrial adenogenesis and also to include dose response studies which, as one of skill in the art will recognize, are routine experiments. An important consideration is that the PRL hormone has a short half-life and therefore no toxicity is expected at high dosage (supra-physiological doses). One could also correlate the effect of neonatal administration of lactogenic and/or somatogenic hormones to the ewe with the fertility of the resulting offspring, as well as with milk production of the mother and the size and vigor of the offspring. Additionally, the administration of the roPRL could be optimized by using a more stable recombinant hormone that would be given less frequently. An alternative to administering the hormone regularly would be to inject directly the animals with a recombinant DNA coding for the hormone and thereby facilitate the expression of the transgene.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

1. Ausubel, F. M. et al. (1992). Current Protocols in Molecular Biology, (John Wiley & Sons, New York, N.Y.).

2. Bartol F F, Wiley A A, Floyd J G, Ott T L, Bazer F W, Gray C A, Spencer T E (1999).

“Uterine differentiation as a foundation for subsequent fertility.” J Reprod Fertil Suppl 54:285-300.

3. Bole-Feysot C, Goffin V, Edery M, Binart N, Kelly P A (1998). “Prolactin (PRL) and its receptor: actions, signal transduction pathways and phenotypes observed in PRL receptor knockout mice.” Endocr Rev 19:225-268.

4. Burton G J, Watson A L, Hempstock J, Skepper J N, Jauniaux E (2002). “Uterine glands provide histiotrophic nutrition for the human fetus during the first trimester of pregnancy.” J Clin Endocrinol Metabol 87: 2954-2959.

5. Chilton B S, Mani S K, Bullock D W (1988). “Servomechanism of prolactin and progesterone in regulating uterine gene expression.” Mol Cell Endocrinol 2:1169-1175.

6. Choi Y, Johnson G A, Burghardt R C, Berghman L R, Joyce M M, Taylor K M, Stewart M D, Bazer F W, Spencer T E (2001). “Interferon regulatory factor two restricts expression of interferon-stimulated genes to the endometrial stroma and glandular epithelium of the ovine uterus.” Biol Reprod 65:1038-1049.

7. Dalrymple A, Jabbour H N (2000). “Localization and signaling of the prolactin receptor in the uterus of the common marmoset monkey.” J Clin Endocrinol Metab 85:1711-1718.

8. Freeman M E, Kanyicska B, Lerant A, Nagy G (2000). “Prolactin: structure, function, and regulation of secretion.” Physiol Rev 80:1523-1563.

9. Gray C A, Johnson G A, Bartol F F, Tarleton B J, Wiley A A, Bazer F W, Spencer T E (2001). “Developmental biology of uterine glands.” Biol Reprod 65:1311-1323.

10. Gray C A, Taylor K M, Bazer F W, Spencer T E (2000). “Mechanisms regulating norgestomet inhibition of endometrial gland morphogenesis in the neonatal ovine uterus.”Mol Reprod Dev 57: 67-78.

11. Gray C A, Taylor K M, Ramsey W S, Hill J R, Bazer F W, Bartol F F, Spencer T E (2001). “Endometrial glands are required for preimplantation conceptus elongation and survival.” Biol Reprod 64:1608-1613.

12. Jabbour H N, Critchley H O, Boddy S C (1998). “Expression of functional prolactin receptors in nonpregnant human endometrium: janus kinase-2, signal transducer and activator of transcription-1 (STAT 1), and STAT 5 proteins are phosphorylated after stimulation with prolactin.” J Clin Endocrinol Metab 83:2545-2553.

13. Jones R L, Critchely H O, Brooks J, Jabbour H N, McNeilly A S (1998). “Localization and temporal expression of prolactin receptor in human endometrium.” J Clin Endocrinol Metab 83:258-262.

14. Kelly M A, Rubinstein M, Asa S L, Zhang G, Saez C, Bunzow J R, Allen R G, Hnasko R, Ben-Jonathan N, Grandy N K, Low M J (1997). “Pituitary lactotroph hyperplasia and chronic hyperprolactinemia in dopamine D2 receptor-deficient mice.” Neuron 19:103-113.

15. Leibovich H, Raver N, Herman A, Gregoraszczuk E L, Gootwine E, Gertler A (2001). “Large-scale preparation of recombinant ovine prolactin and determination of its in vitro and in vivo activity.” Protein Expr Purif 22:489-496.

16. Sambrook, J. et al. (1989). Molecular Cloning: A Laboratory Manual, 2nd Ed. (Cold Spring Harbor Laboratory, Cold Spring Harbor, N.Y.).

17. SAS. SAS User's Guide: Statistics, (1990) Version 6. Cary, N.C.: Statistical Analysis System Institute, Inc.

18. Spencer T E, Bartol F F, Bazer F W, Johnson G A, Joyce M M (1999). “Identification and characterization of glycosylation dependent cell adhesion molecule 1 (GlyCAM-1) expression in the ovine uterus.” Biol Reprod 60: 241-250.

19. Spencer T E, Stagg A G, Taylor K M, Johnson G A, Gertler A, Gootwine E, Bazer F W (1999). “Effects of recombinant ovine interferon tau, placental lactogen and growth hormone on ovine endometrial function.” Biol Reprod 61:1409-1418.

20. Taylor K M, Gray C A, Joyce M M, Stewart M D, Bazer F W, Spencer T E (2000). “Neonatal ovine uterine development involves alterations in expression of receptors for estrogen, progesterone, and prolactin.” Biol Reprod 63:1192-1204.

21. Taylor K M, Chen C, Gray C A, Joyce M M, Bazer F W, Spencer T E (2001). “Expression of mRNAs for fibroblast growth factors 7 and 10, hepatocyte growth factor and insulin-like growth factors and their receptors in the neonatal ovine uterus.” Biol Reprod 64:1236-1246.

22. Young K H, Kraeling R R, Bazer F W (1989). “Effects of prolactin on conceptus survival and uterine secretory activity in pigs.” J Reprod Fertil 86:713-722.

23. Yu-Lee L (2001). “Stimulation of interferon regulatory factor-I by prolactin.” Lupus 10:691-699.

What is claimed is:

1. A method for increasing the reproductive efficiency of a mammal comprising:
administering an effective amount of at least one lactogenic hormone to increase reproductive efficiency of the mammal.

2. The method of claim 1, further comprising administering an effective amount of at least one somatogenic hormone to the mammal.

3. The method of claim 1, wherein the lactogenic hormone is prolactin or placental lactogen.

4. The method of claim 2, wherein the somatogenic hormone is growth hormone.

5. The method of claim 1, wherein the hormone is recombinant.

6. The method of claim 1, wherein the hormone is exogenous.

7. The method of claim 1, wherein the administering is to a uterus.

8. The method of claim 1, wherein the mammal is a human, a primate, or an ungulate.

9. The method of claim 1, wherein the hormone is administered to a neonatal mammal.

10. The method of claim 1, wherein the hormone is administered in utero to an embryo.

11. The method of claim 3, wherein the effective amount of lactogenic hormone is about 2 milligrams per kilogram body weight per day.

12. The method of claim 3, wherein the hormone is a recombinant ovine prolactin.

13. The method of claim 9, wherein the hormone is administered from birth to 90 days postnatally.

14. The method of claim 13, wherein the hormone is administered from birth to 60 days postnatally.

15. The method of claim 7, wherein the uterus has been subjected to at least one assisted reproductive technology.

16. The method of claim 15, wherein the assisted reproductive technology is in vitro fertilization followed by embryo transfer (IVF-ET).

17. The method of claim 7, wherein the uterus is an ungulate uterus and the hormone is a recombinant ovine prolactin.

18. A method for increasing endometrial adenogenesis in a mammal comprising: administering an effective amount of at least one lactogenic hormone to a mammal to increase endometrial adenogenesis.

19. The method of claim 18, further comprising administering an effective amount of at least one somatogenic hormone to the mammal.

20. The method of claim 18, wherein the lactogenic hormone is prolactin or placental lactogen.

21. The method of claim 19, wherein the somatogenic hormone is growth hormone.

22. The method of claim 18, wherein the hormone is recombinant.

23. The method of claim 18, wherein the hormone is exogenous.

24. The method of claim 18, wherein the administering is to a uterus.

25. The method of claim 18, wherein the mammal is a human, a primate, or an ungulate.

26. The method of claim 18, wherein the hormone is administered to a neonatal mammal.

27. The method of claim 18, wherein the hormone is administered in utero to an embryo.

28. The method of claim 20, wherein the effective amount of lactogenic hormone is about 2 milligram per kilogram body weight per day.

29. The method of claim 20, wherein the hormone is a recombinant ovine prolactin.

30. The method of claim 26, wherein the hormone is administered from birth to 90 days postnatally.

31. The method of claim 30, wherein the hormone is administered from birth to 60 days postnatally.

32. The method of claim 24, wherein the uterus has been subjected to at least one assisted reproductive technology.

33. The method of claim 32, wherein the assisted reproductive technology is in vitro fertilization followed by embryo transfer (IVF-ET).

34. The method of claim 24, wherein the uterus is an ungulate uterus and the hormone is a recombinant ovine prolactin.

35. A method for increasing the reproductive efficiency of an adult mammal comprising: administering an effective amount of at least one lactogenic or somatogenic hormone to increase reproductive efficiency of the adult mammal.

36. The method of claim 35, further wherein an effective amount of at least one lactogenic and at least one somatogenic hormone is administered to the adult mammal to increase its reproductive efficiency.

37. The method of claim 35, wherein the lactogenic hormone is prolactin or placental lactogen.

38. The method of claim 35, wherein the somatogenic hormone is growth hormone.

39. The method of claim 35, wherein the hormone is recombinant.

40. The method of claim 35, wherein the hormone is exogenous.

41. The method of claim 35, wherein the administering is to a uterus.

42. The method of claim 35, wherein the mammal is a human, a primate, or an ungulate.

43. The method of claim 37, wherein the hormone is a recombinant ovine prolactin.

44. The method of claim 37, wherein the effective amount of lactogenic hormone is 2 milligrams per kilogram body weight per day.

45. The method of claim 41, wherein the uterus has been subjected to at least one assisted reproductive technology.

46. The method of claim 45, wherein the assisted reproductive technology is in vitro fertilization followed by embryo transfer (IVF-ET).

47. The method of claim 41, wherein the uterus is an ungulate uterus and the hormone is a recombinant ovine prolactin.
